(19) United States
(12) Patent Application Publication
(10) Pub. No.: US 2021/0272683 A1
(43) Pub. Date: Sep. 2, 2021

(54) RADIOMETRY SYSTEMS AND METHODS FOR DENTAL APPLICATIONS

(71) Applicant: AdDent, Inc., Danbury, CT (US)

(72) Inventor: Joshua Friedman, Ridgefield, CT (US)

(73) Assignee: AdDent, Inc., Danbury, CT (US)

(21) Appl. No.: 17/321,388

(22) Filed: May 14, 2021

Related U.S. Application Data
(63) Continuation-in-part of application No. 16/416,896, filed on May 20, 2019, now abandoned.
(60) Provisional application No. 62/815,879, filed on Mar. 8, 2019.

(51) Int. Cl. G16H 40/20; G16H 20/40; G16H 40/67; G06K 7/14; G06K 7/10; G16H 10/60; A61C 13/15

(52) U.S. Cl. CPC G16H 40/20; G16H 20/40; G16H 40/67; G06K 7/1413; G16H 40/40; G06K 7/10297; G16H 10/60; A61C 19/004; G06K 2007/10504; G06K 7/1417

(57) ABSTRACT

The present disclosure describes systems and methods for radiometry in dental applications. The disclosed systems include a radiometer, a dental curing light, a composite material reader, and a restoration data storage device, where one or more of the radiometer, dental curing light, composite material reader, and restoration data storage device include one or more communication modules that enable wireless communication between one or more components. The radiometer is preferably programmed to determine the change in a rate of cure of a light-curable composite material and thereby determine whether an optimum cure time has been reached for the light-curable composite material. The communication modules may be used to transmit information between the radiometer and the curing light, and between one or more of the radiometer and curing light and one or more of the composite material reader and restoration data storage device.

### CROSS-REFERENCE TO RELATED APPLICATIONS

This application is a continuation-in-part of U.S. patent application Ser. No. 16/416,896, filed on May 20, 2019, which claims the benefit of and priority to U.S. Provisional Patent Application Ser. No. 62/815,879, filed on Mar. 8, 2019, the entireties of which are hereby incorporated herein by reference.

### BACKGROUND

### Field of the Invention

The present disclosure relates to radiometers and curing lights for use in dental applications.

### Description of the Related Art

Dental curing lights have been used for over three decades to polymerize composite materials for use as dental fillings. A dental clinician places an unpolymerized composite, material in a patient's mouth and configures it according to clinical needs, and then subsequently rapidly polymerizes the material using, the curing light so that it becomes a rigid dental filling. The basic types of dental curing light sources are tungsten halogen, light-emitting diode (LED), plasma arc, and laser.

The use of a radiometer in combination with a curing light allows a dental clinician to measure the light output of the curing light. A number of factors determine the degree of polymerization, including (1) the intensity of the curing light, (2) the depth of the restoration, (3) the shade of the composite material, (4) the type of filler and the chemistry of the composite material, (5) the age of the material, and (6) the wavelength of the light applied for curing.

U.S. Pat. No. 7,175,436 to Friedman (“the '436 patent”) discloses a radiometer and a method for providing an indication of the amount of time needed to cause a light-curable dental resin composite material to optimally polymerize in response to the application of light from any light-curing source during the preparation of as dental restoration. However, the '436 patent does not include a component for communication between the radiometer and curing light used therewith or a method for electronically tracking information regarding the restorative material used to treat a specific patient.

Thus there remains a need for a dental radiometry system that includes a component for communication between a radiometer and a curing light used therewith and that allows electronic transmission of information related to the restorative material used in a given restoration.

### SUMMARY

The present disclosure describes systems and methods for radiometry in dental applications. The disclosed systems include a radiometer, a dental curing light, a composite material reader, and a restoration data storage device, where one or more of the radiometer, dental curing light, composite material reader, and restoration data storage device may include one or more communication modules that enable wireless communication between one or more of the radiometer, curing light, composite material reader, and restoration data storage device. The one or more communication modules may be one or more Bluetooth, Wi-Fi, ZigBee, or other radio frequency based modules. In some highly preferred embodiments, each of the radiometer, curing light, composite material reader module, and restoration data storage device is configured to wirelessly transmit or receive information via Bluetooth, Wi-Fi, or ZigBee. In alternative embodiments, the one or more communication modules may be one or more infrared or other optically-based modules. In some preferred embodiments, the radiometer has at least one communication module and the curing light also has at least one communication module. In such embodiments, the communication modules may be used to transmit information between the radiometer and the curing light, and between one or more of the radiometer and curing light and one or more of the composite material reader and restoration data storage device. In some embodiments, additional communication modules may also act to relay instructions between other communication modules, such as to extend the range of communication, or to convert transmissions between different formats, such as Wi-Fi to Bluetooth or ZigBee to infrared.

In some embodiments, the composite material reader may be a bar code reader, QR code reader, NFC tag reader, or any other similar device. The composite material reader may allow a user to obtain information from a package or container of a composite material that is, labeled with a readable code, chip, mark, or tag such as by a bar code, QR code, or NFC tag. In some alternative embodiments, the composite material reader may be an image scanner that can detect text or images to obtain information from a label associated with a package or container of a composite material.

In some embodiments, the restoration data storage device may be a computer configured to receive information from the radiometer. The restoration data storage device may preferably have one or more communication modules that are configured to wirelessly communicate with at least the communication module of the radiometer. The restoration data storage device communication modules may be one or more Bluetooth, Wi-Fi, ZigBee, or other radio frequency-based modules. In alternative embodiments, the one or more restoration data storage device communication modules may be one or more radio frequency, infrared, or other optically-based modules.

Methods of using the disclosed systems to optimally cure a light-curable composite material are also disclosed herein.

### BRIEF DESCRIPTION OF THE DRAWINGS

The figures provided herewith are intended to illustrate but not to limit the invention. Reference numbers are re-used in the figures to indicate correspondence between referenced elements,

FIG. 1 shows a block diagram of an embodiment of the disclosed systems.

FIG. 2 shows a block diagram of an embodiment of the radiometer used in the disclosed systems.

FIG. 3a shows a housing assembly of an embodiment of the radiometer used in the disclosed systems, wherein a sample holder for holding a test sample of light-curable material is shown separated from the radiometer adjacent to a light guide for a standard light source.

FIG. 3b shows a view of the sample holder shown in FIG. 3a.

FIG. 3c shows another view of the sample holder shown in FIG. 3a.

FIG. 4 shows an embodiment of the radiometer and curing light used in the disclosed systems.

These and other features, aspects, and advantages are described below with reference to the drawings, which are intended to illustrate but not to limit the invention. In the drawings, like reference characters denote corresponding features consistently throughout similar embodiments.

### DETAILED DESCRIPTION

The present disclosure describes systems and methods for radiometry in dental applications. The disclosed systems include a radiometer, a dental curing light, a composite material reader, and a restoration data storage device, where one or more of the radiometer, dental curing light, composite material reader, and restoration data storage device may include one or more communication modules that enable wireless communication between one or more of the radiometer, curing light, composite material reader, and restoration data storage device. The one or more communication modules may be one or more Bluetooth, Wi-Fi, ZigBee, or other radio frequency-based modules. In some highly preferred embodiments, each of the radiometer, curing light, composite material reader module, and restoration data storage device is configured to wirelessly transmit or receive information via Bluetooth, Wi-Fi, or ZigBee. In alternative embodiments, the one or more communication modules may be one or more infrared or other optically-based modules. In some preferred embodiments, the radiometer has at least one communication module and the curing light also has at least one communication module. In such embodiments, the communication modules may be used to transmit information between the radiometer and the curing light, and between one or more of the radiometer and curing light and one or more of the composite material reader and restoration data storage device. In some embodiments, additional communication modules may also act to relay instructions between other communication modules such as to extend the range of communication, or to convert transmissions between different formats, such as Wi-Fi to Bluetooth or ZigBee to infrared.

For example, the communication modules described herein may be composed of a Bluetooth-enabled network component that initiates or joins a Bluetooth network to communicate with other communication modules. In this manner, the radiometer, the dental curing light, the composite material reader, and the restoration data storage device may then be able to exchange data, such as sensor readings, instructions, and so on via such a Bluetooth network.

In some embodiments, the radiometer may further include a microprocessor and a memory module, where the microprocessor is configured to record information for storage on the memory module. In such embodiments, the one or more communication modules of the radiometer are preferably configured to obtain information stored on the memory module and wirelessly transmit said information. For example, the one or more communication modules of the radiometer may transmit sensor data via the Bluetooth network to an application that is connected to the Bluetooth network via a personal computer or a smart device, such as a phone or tablet.

In some embodiments, the composite, material reader may be a bar code reader, QR code reader, NFC tag reader, or any other similar device. The composite material reader may allow a user to Obtain information from a package or container of a composite material that is labeled with a readable code, chip, mark, or tag such as by a bar code, QR code, or NFC tag. In some alternative embodiments, the composite material reader may be an image scanner that can detect text or images to obtain information from a label associated with a package or container of a composite material.

In such embodiments, the one or more communication modules of the composite material reader are preferably configured to obtain information stored on the composite material reader and wirelessly transmit said information. For example, the one or more communication modules of the radiometer may transmit any information obtained from a readable code, chip, mark, or tag by the composite material reader (such as by a bar code, QR code, or NFC tag) via the Bluetooth network to an application that is connected to the Bluetooth network via a personal computer or a smart device, such as a phone or tablet.

In some embodiments, the restoration data storage device may be a computer, such as a personal computer or smart device, that is configured to receive information from the radiometer. The restoration data storage device may preferably have one or more communication modules that are configured to wirelessly communicate with at least the communication module of the radiometer. The restoration data storage device communication modules may be one or more Bluetooth, Wi-Fi, ZigBee, or other radio frequency-based modules. In alternative embodiments, the one or more restoration data storage device communication modules may be one or more radio frequency, infrared, or other optically -based modules.

Methods of using the disclosed systems to optimally cure a light-curable composite material are also disclosed herein.

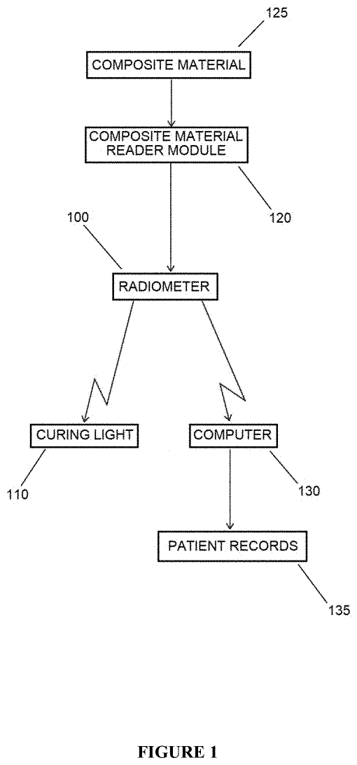

FIG. 1 shows a block diagram of an embodiment of the disclosed systems. The embodiment shown in FIG. 1 includes a radiometer 100, a curing light 110, a composite material reader 120, and a computer 139, where each component includes one or more communication modules (not shown) configured to transmit information between the components. A package of composite material 125 may have information stored on a readable code, chip, mark, or tag such as a bar code, QR code, or NFC tag. The composite material reader 120 is configured to obtain information from the package of composite material 125 and is also configured to transmit information to the radiometer 100. In some embodiments, composite material reader 120 transmits information to the radiometer 100 indirectly, such as through computer 130. The radiometer 100 is configured to transmit information to the curing light 110 and to the computer 130. In some embodiments, radiometer 100 transmits information to curing light 110 indirectly, such as through computer 130, The computer 130 may optionally store information received from the radiometer 100 in a patient records database 135.

### Radiometer

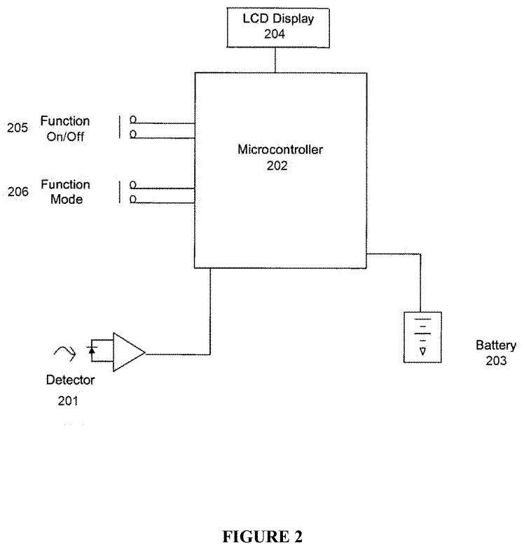

In some preferred embodiments, the disclosed systems may include a radiometer disclosed in U.S. Pat. No. 7,175,435 to Friedman (“the '436 patent”) or a similar radiometer that includes additional features or removes features as needed for optimum use in the disclosed systems. As used herein, the term “radiometer” refers to a radiometer that is capable of monitoring the change in intensity of light traveling through a material analogous to the monitoring process in Fourier transform infrared (FTIR) spectroscopy, in some highly preferred embodiments, the radiometer is programmed to determine the change in a rate of cure of a light-curable composite material and thereby determine whether an optimum cure time has been reached for the light-curable composite material. It will be understood by ordinary skilled artisans that the rate of cure of a light-curable composite material corresponds to the degree of polymerization of the material. An embodiment of the radiometer 200 is shown in FIGS. 2, 3, and 4.

FIG. 2 represents a block diagram of the internal electronic components of the radiometer 200. Accordingly, the radiometer 290 includes a detector cell 201 for providing either an output voltage or a change in electrical resistance in direct response to the degree of light exposure. The detector cell 201 may be composed of a light sensor such as a silicon, CMOS, or selenium detector cell or another light sensor. In addition, the radiometer 200 limber includes a microcontroller (microprocessor) 202, a battery 203, a display 204, an on/off function switch 205, and a mode switch 296. The display 204 may preferably be an LCD display. The mode switch 206 may be used to toggle between different modes of use of the radiometer 200. In some embodiments, the radiometer may be used in an “Optical Conversion” mode, a “Power” mode, an “Energy” mode, or a “Calibration” mode. When Optical Conversion mode is selected, the display 204 may provide a time display output that indicates the shortest exposure time to provide optimal composite cure for a sample of uncured composite using any type of light source. In some embodiments, the mode may be selected via an instruction received by the one or more communication modules of the radiometer 200. For example, an application on computer 130 may display a menu allowing a user to select one or more modes of operation, which is then transmitted as an instruction from computer 130 to radiometer 290.

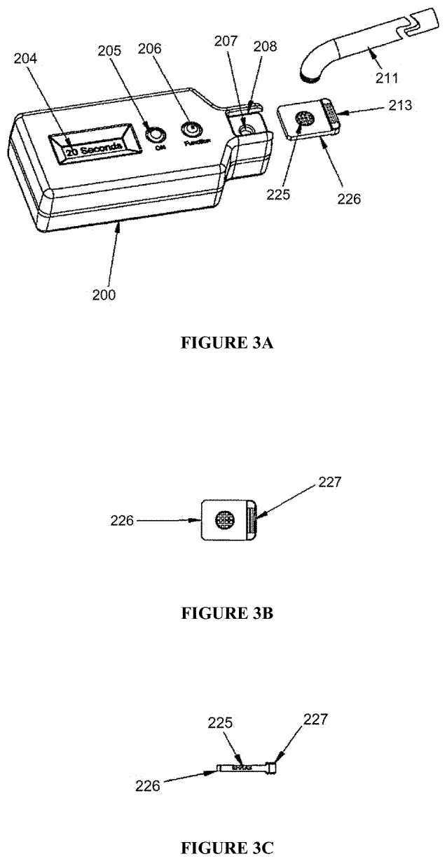

FIG. 3a shows a housing assembly of an embodiment of the radiometer used in the disclosed systems, wherein a sample holder for holding a test sample of light-curable material is shown separated from the radiometer adjacent to a light guide for a standard light source. FIGS. 3b and 3c show views of the sample holder shown in FIG. 3a.

In Optical Conversion mode, a light curing source (not shown) with a light guide 211 may be used to cure a sample of an uncured light-curable composite material 225 as described below. A sample of uncured light-curable composite material 225 is placed in a sample bolder 226 of appropriate thickness for a given restoration. The sample holder 226 may have a thickness that corresponds to a typical required depth of a dental restoration, and thus by varying the thickness of the sample holder 226, the thickness of the sample 225 may be adjusted. The sample 225 is held by a grip detail 227, as shown in FIG. 3c, and is inserted along a groove or track 208, as shown in FIG. 3a, so that the sample sits directly over the detector window 207 of the light sensor. The light guide 211 is placed over the sample aligned with the detector window 207 so that light may be transmitted through the sample 225. Optical Conversion mode may then be selected using the mode switch 206. Alternatively, Optical Conversion mode may be selected via an application as described herein. The display 204 displays the time needed to maximally cure the composite, i.e., curing will be stopped when the display shows a time corresponding to the exposure duration needed to achieve the composite cure for the sample composite that represents a time when the sample is cured in accordance with the algorithm used in programming the microcontroller 202. The microcontroller 202 may be programmed using an algorithm such as the algorithm disclosed in the '436 patent. The extent of curing of the composite material may be 80-99.5% of the maximum possible cure value. For most composite resin materials, the extent of composite cure will plateau at 45-70% of the maximum cure value (100%) tips the material. The sampling rate used by the microcontroller 202 may preferably be less than or equal to 0.1 Hz.

In some embodiments, the algorithm may be programmed via instructions received by the one or more communication modules of the radiometer 200. For example, an application running on computer 130 may receive an updated algorithm which it then sends to radiometer 200 via their associated communication modules. Upon receipt of the algorithm via its communication module, radiometer 200 may then update microcontroller 202 with the new algorithm. In some embodiments, any information displayed by the radiometer 200 may be sent via the one or more communication modules to another device. For example, the time needed to maximally cure the composite may be sent via the one or more communication modules of radiometer 200 to computer 130, wherein it may then be displayed in an application to a user.

In Power mode, the radiometer 200 measures the curing light output intensity. The intensity may preferably be displayed in W/cm² or mW/cm². The display 204 may preferably be programmed to update the displayed output intensity as long as the mode switch 206 is activated. The mode switch 206 may, for example, be activated when a push button is depressed. When the mode switch 206 is deactivated, such as by releasing a push button, the radiometer will continue to measure the curing light output intensity but the display will correspond only to the peak measurements. This mode of using a radiometer is also termed “irradiance” in various references. In some embodiments, the curing light output intensity may be sent via the one or more communication modules of radiometer 200 to computer 130, wherein it may then be displayed in an application to a user.

In Energy mode, the accumulated energy delivered to a composite material may be measured. The measurement may preferably be displayed in J or mJ. Activating the mode switch 206 may reset the measurement. In some embodiments, the accumulated energy measurements may be sent via the one or more communication modules of radiometer 200 to computer 130, wherein it may then be displayed in an application to a user.

In Calibration mode, the radiometer may be calibrated using a standard light source and a calibration filter that has the same or nearly the same optical transmission characteristics as a fully cured dental composite material. The calibration filter may preferably comprise a polymer material. The exposure time displayed may then be compared using the calibration filter and the light unit being tested. The microcontroller may be programmed to adjust the offset if a given sequence of switches is activated simultaneously or serially. In some embodiments, the exposure time or other calibration information may be sent via the one or more communication modules of radiometer 200 to computer 130, wherein it may then be displayed in an application to a user.

The on/off function switch 205 may be used to turn on or turn off the radiometer 200. In some embodiments, the radiometer 200 may be programmed to automatically turn off or enter a low power state if it is unused for a specified period of time. In additional embodiments, the radiometer 200 may be programmed to automatically turn off or enter a low power state if it is unused for a specified period of time based on instructions received from the one or more communication modules of radiometer 200, such as instructions sent via an application on computer 130.

The display 204 may display real-time light intensity (power density), accumulated light energy delivered, or recommended exposure time depending on the mode of operation. The display 204 may preferably be an LCD display. In some embodiments, real-time light intensity (power density), accumulated light energy delivered, or recommended exposure time depending on the mode of operation may be transmitted by the communication modules of radiometer 290 for display on another device, such as on an application on computer 130.

In some embodiments, the light sensor may be a solid-state photo detector.

The radiometer may preferably be battery-operated.

FIG. 3b shows a view of the sample bolder 226 shown in FIG. 3a, including the grip detail 227.

FIG. 3c shows another view of the sample holder 226 shown in FIG. 3a, including the grip detail 227 and the sample 225 loaded therein.

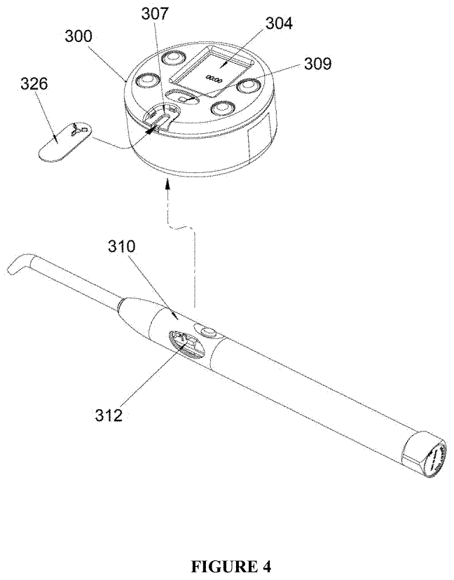

FIG. 4 shows an embodiment of a radiometer 300 and curing light 310 used in the disclosed systems. A clinician inserts a sample of composite material into a sample holder 326. In some embodiments, the sample holder 326 may preferably have a depth of 2, 4, or 6 mm to accommodate dental restorations of various desired depths. The sample holder 326 is inserted into an optical reader port 307 of the radiometer 300. Using the same curing light and composite material to be used in the restoration procedure, a clinician polymerizes the sample composite material. As polymerization occurs, the minimum optimal cure time is determined by the radiometer as described in the '436 patent. The time is displayed on the display 304. The communication module 309 enables transmission of the optimal cure time information from the radiometer to the curing light, and transmission of this and other information between the radiometer and other components of the system. The communication module 309 preferably includes an on-board memory module with sufficient memory to store the information that will be transmitted from the radiometer to other components of the system. In some embodiments, the radiometer 200 may be programmed to send an instruction to the curing light when the optimum cure time has been reached and the curing light may be programmed to automatically turn off immediately upon receipt of said instruction. In some embodiments, radiometer 200 may also transmit the curing time to another device for display, such as computer 130. In addition, radiometer 200 may also transmit information sufficient for another device, such as computer 130, to determine the start of curing, the time remaining or estimated time until curing is complete, or the end of curing. A device may also be able to send instructions to radiometer 200 via its one or more communication modules with respect to curing. For example, computer 130 may be able to send instructions to extend the curing time.

### Curing Light

The disclosed systems further include a dental curing light. The dental curing light may be used to cure a light-curable composite material. The curing light may preferably have a light guide. The curing light may include at least one communication module that allows transmission of information from the radiometer to the curing light.

FIG. 4 shows an embodiment of a curing light 310 used in the disclosed systems, including a communication module 312. The communication module may be configured to receive information from the radiometer regarding the optimum cure time for the composite material being used in a given restoration. In other embodiments, the communication module may be configured to receive from information regarding the optimum cure time from another device, such as computer 130. In some embodiments, the curing light may be programmed to automatically turn off once the optimum cure time has been reached.

In some embodiments, the radiometer may be programmed to send an instruction to the curing light when the optimum cure time has been reached, and the curing light may be programmed to automatically turn off immediately upon receipt of said instruction.

In some preferred embodiments, the radiometer does not control the intensity of the light generated by the curing light.

### Communication Modules

One or more components of the disclosed systems, namely the radiometer, curing light, composite material, reader, and restoration data storage device, may include one or more communication modules that enable wireless communication between the radiometer, curing light, composite material reader, and restoration data storage device. The one or more communication modules may be one or more Bluetooth, Wi-Fi, ZigBee, or other radio frequency-based modules. In alternative embodiments, the one or more communication modules may be one or more infrared or other optically-based modules for sending or receiving information as described herein. Each of the one or more communication modules preferably includes an on-board memory module with sufficient memory to store the information that may be transmitted between the communication module and other communication modules or other components of the system, including both information that will be transmitted by the communication module and information that will be received by the communication module. In some preferred embodiments, the radiometer may include at least one communication module and the curing light also may include at least one communication module. In such embodiments, the communication modules may be used to transmit information between the radiometer and the curing light, and between one or more of the radiometer and curing light and one or more of the composite material reader and restoration data storage device described below. In some embodiments, additional communication modules may also act to relay instructions between other communication modules, such as to extend the range of communication, or to convert transmissions between different formats, such as Wi-Fi to Bluetooth or ZigBee to infrared.

In some embodiments, the minimum amount of time required to optimally cure an uncured light-curable composite material may be wirelessly transmitted from the radiometer to the curing light using the communication modules of the radiometer and curing light respectively. The curing light may preferably be configured to automatically turn off at the time at which the light-curable composite material in use has been optimally cured. In other embodiments, a visual or audio indicator, such as a flickering LED or a musical chime, may be used to indicate when the light-curable composite material in use has been optimally cured. The optimal curing time may be determined by the change in the rate of cure, which depends on factors such as (1) the intensity of the curing light, (2) the depth of the restoration, (3) the shade of the composite material, (4) the type of filler and the chemistry of the composite material, (5) the age of the material, and (6) the wavelength of the light applied for curing.

### Composite Material Reader

The disclosed systems further include a composite material reader. The composite material reader may be a bar code reader, QR code reader, NFC tag reader, or any other similar device. The composite material reader may allow a user to obtain information from a package or container of a light-curable composite material that is labeled with a readable code, chip, mark, or tag such as by a bar code, QR code or NFC tag. In some alternative embodiments, the composite material reader may be an image scanner that can detect text or images to obtain information from a label associated with a specific package or container of aa composite material.

In some embodiments, information that may be provided on the readable code, chip, mark, or tag of a package or container of a light-curable composite material may include the type of material, manufacturer, serial number, lot code number, use-by date or expiration date, and specification information for the material such as the shade of the material and other relevant information.

The composite material reader may preferably have one or more communication modules. The one or more composite material reader communication modules may be one or more Bluetooth, Wi-Fi, Zigbee, or other radio frequency-based modules. In alternative embodiments, the one or more composite material reader communication modules may be one or more infrared or other optically-based modules. In some embodiments, the one or more composite material reader communication modules may transmit data to the radiometer, such as any information obtained from a readable code, chip, mark, or tag. In alternate embodiments, the one or more composite material reader communication modules may transmit data, such as any information obtained from a readable code, chip, mark, or tag, directly to the restoration data storage device described below.

Information transmitted from the composite material reader's one or more communication modules to the radiometer's one or more communication modules or restoration data storage device's communication modules described below may include the type of material, manufacturer, serial number, lot code number, use-by date or expiration date, and specification information for the material such as the shade of the material and other relevant information.

In some embodiments, the information transmitted from the composite material reader may be used by the radiometer or restoration data storage device to determine a curing time, an algorithm for curing, or to perform compliance tests, For example, if the material is past its expiration date, an application on the restoration data storage device or the radiometer may indicate the need to replace the material. As another example, the shade of the material may be compared by such an application or the radiometer with patient data to ensure that proper color matching with the patient's teeth is obtained. As yet another example, information such as type of material and manufacturer may be checked by such an application or the radiometer to determine appropriate insurance coverage. In addition, such an application or the radiometer may use such information to check a database for recalls or other potential issues.

In some embodiments, the composite material reader may preferably be a digital scanner.

### Restoration Data Storage Device

The disclosed systems further include a restoration data storage device. In some embodiments, the restoration data storage device may be a computer configured to send or receive data from the radiometer. The restoration data storage device may preferably have one or more communication modules that are configured to wirelessly communicate with at least the communication module of the radiometer. The one or more restoration data storage device communication modules may be one or more Bluetooth, Wi-Fi, ZigBee, or other radio frequency-based modules. In alternative embodiments, the one or more restoration data storage device communication modules may be one or more infrared or other optically-based modules.

Information transmitted from the one or more radiometer communication modules to the one or more restoration data storage device communication modules may include the depth of the restoration and the curing time.

Information transmitted from the one or more composite material reader communication modules to the one or more restoration data storage device communication modules may include the type of material, manufacturer, serial number, lot code number, use-by date or expiration date, and specification information for the material such as the shade of the material and other relevant information. In some embodiments, this information may be transmitted from the one or more composite material reader communication modules to the one or more radiometer communication modules and then from the one or more radiometer communication modules to the one or more restoration data storage device communication modules. In alternate embodiments, this information may be transmitted directly from the one or more composite material reader communication modules to the one or more restoration data storage device communication modules.

Additional information recorded by the restoration data storage device may include the name of the patient, the date of placement of the restoration, and the number and type of surfaces restored (also known as the class of the restoration).

In some embodiments, the restoration data storage device may include an application running on a personal computer or smart device, such as a phone or tablet. In such embodiments, information displayed on other devices, such as the radiometer 200, may be transmitted to the restoration data storage device for display by the application, in addition, with respect to operations that may be applied to other devices as described herein, such operations may be applied indirectly via the application running on the restoration data storage de ice. For example, the application may instruct the composite material reader to read a tag via the application, instruct the radiometer to select a specific mode, start a curing process, etc.

### Advantages of Using Disclosed Systems

Wireless transmission of data using the disclosed systems provides numerous advantages for clinicians performing dental restorations. Wireless transmission of data reduces or eliminates the possibility of human error in recording the appropriate curing time or other relevant information. It allows dental clinicians to focus on patient treatment rather than data recordation. In addition, in the event of a future issue with a dental restoration such as breakage, discoloration, unusual wear, or an allergic reaction, a dental clinician would be able to access specific details regarding the restoration, including the type of material used, that would inform decisions regarding resolution of the issue. Moreover, by providing manufacturers of composite materials with information regarding the specific successes and failures of specific restorations, manufacturers will be better informed and able to reach more accurate and specific conclusions regarding issues such as whether a particular lot, of material was defective or whether a particular use-by date is appropriate for a given composite material. In addition a manufacturer would be able to communicate information regarding a defective batch or lot of material and clinicians would be able to identify necessary remedial measures on a patient-by-patient basis.

Wireless transmission of data using, the disclosed systems may also be used to allow a computer to update and adapt a radiometer, a curing light, or a composite material reader to operate within the disclosed systems. For example, a firmware upgrade may be pushed out by a computer. As another example, a computer may send instructions to a curing light restricting operation of the curing light to prevent a user from damaging a selected composite material. As yet another example, the curing light output may be monitored or controlled using a handheld computer, such as a smartphone or tablet.

The previous description of the disclosed embodiments is provided to enable any person skilled in the art to make or use the invention disclosed herein. Although the various inventive aspects are disclosed in the context of certain illustrated embodiments, implementations, and examples, it should be understood by those skilled in the art that the invention extends beyond the specifically disclosed embodiments to other alternative embodiments and/or uses of the invention and obvious modifications and equivalents thereof. In addition, while a number of variations of various inventive aspects have been shown and described in detail, other modifications that are within their scope will be readily apparent to those skilled in the art based upon reviewing this disclosure. It should be also understood that the scope of this disclosure includes the various combinations or sub-combinations of the specific features and aspects of the embodiments disclosed herein, such that the various features, modes of implementation, and aspects of the disclosed subject matter may be combined with or substituted for one another. The generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Similarly, the disclosure is not to be interpreted as reflecting an intent that any claim set forth below requires more features than are expressly recited in that claim. Rather, as the following claims reflect, inventive aspects may reside in a combination of fewer than all features of any single foregoing disclosed embodiment.

Each of the foregoing and various aspects, together with those set forth in the claims and summarized above or otherwise disclosed herein, including the figures, may be combined without limitation to form claims for a device, apparatus, system, method of manufacture, and/or method of use.

All references cited herein are hereby expressly incorporated by reference.

What is claimed is:

1. A radiometry system for use in dental applications comprising:
a. a radiometer comprising a detector cell, a microprocessor, a memory module, and a first set of one or more communication modules;
b. a curing light that is used to generate light and that includes a second set of one or more communication modules;
c. a composite material reader that includes a third set of one or more communication modules; and
d. a restoration data storage device that includes a fourth set of one or more communication modules;
wherein the microprocessor is configured to record information for storage on the memory module;
wherein the radiometer is configured to send instructions to the curing light via the first set of one or more communication modules;
wherein the curing light is configured to receive instructions via the second set of one or more communication modules;
wherein the composite material reader is configured to obtain a set of information from a package or container that contains a light-curable composite material and to send the set of information via the third set of one or more communication modules;
wherein the intensity of the light generated by the curing light is not controlled by the radiometer; and
wherein the radiometer is programmed to determine the change in a rate of cure of a light-curable composite material and thereby determine whether an optimum cure time has been reached for the light-curable composite material.

2. The system of claim 1 wherein the first set of one or more communications modules, the second set of one or more communication modules, the third set of one or more communication modules, and the fourth set of one or more communication modules are configured to transmit and receive information to and from the radiometer, the curing light, the composite material reader module, or the restoration, data storage device.

3. The system of claim 3 wherein the radiometer is programmed to send an instruction to the curing light when the optimum cure time has been reached and the curing light is programmed to automatically turn off immediately upon receipt of said instruction.

4. The system of claim 1, wherein at least one of the radiometer or restoration data storage device receives the set of information regarding the package or container.

5. The system of claim 5 wherein the composite material reader includes a component that is selected from the group consisting of a bar code reader, a QR code reader, and an NFC tag reader.

6. The system of claim 5 wherein the composite material reader includes a east one optical scanner.

7. The system of claim 5 wherein the first, second, third, and fourth sets of one or more communication modules are each selected from the group consisting of Bluetooth, Wi-Fi, and ZigBee modules.

8. A radiometry system for use in dental applications comprising:
a. a radiometer comprising a detector cell, a microprocessor, and a memory module;
b. a curing light that is used to generate light;
c. a composite material reader and
d. a restoration data storage device;
wherein each of the radiometer, curing light, composite material reader, and restoration data storage device is configured to wirelessly transmit or receive information via Bluetooth, Wi-Fi, or ZigBee;
wherein the microprocessor is configured to record information for storage on the memory module;
wherein the intensity of the light generated by the curing light is not controlled by the radiometer; and
wherein the radiometer is programmed to determine the change in a rate of cure of a light-curable composite material and thereby determine whether an optimum cure time has been reached for the light-curable composite material.

10. The system of claim 9 wherein the information includes a first set of information obtained from a package or container that contains a light-curable composite material.

11. The system of claim 10 wherein the radiometer is programmed to send an instruction to the curing light when the optimum cure time has been reached and the curing light is programmed to automatically turn off immediately upon receipt of said instruction.

12. The system of claim 11 wherein the composite material reader includes a component that is configured to obtain the first set of information from the package or container that contains a light-curable composite material.

13. Ihe system of claim 12 wherein the composite material reader includes a component that is selected from the group consisting of a bar code reader, a QR code reader, and an NFC tag reader.

14. The system of claim 12 wherein the composite material reader includes at least one optical scanner.

15. The system of claim 12, wherein the composite material reader determines and wirelessly transmits the first set of information, and wherein the first set of information includes one or more of:
a. a depth of the restoration; and
b. a curing time.

16. The system of claim 15, wherein the composite material reader determines and wirelessly transmits the first set of information, and wherein the first set of information further includes one or more of:
c. a type of material;
d. a manufacturer of the material;
e. a serial number of the material;
f. a lot code number of the material;
g. a use-by date or expiration date of the material; and
h. specification information for the material.

17. The system of claim 16 wherein the restoration data storage device is configured to obtain a second set of information, wherein the second set of information includes one or more of:
i. a name of a patient;
j. a date of placement of restoration; and
k. a number and type of restored surfaces.
